(19) United States
(12) Patent Application Publication
(10) Pub. No.: US 2010/0042150 A1
(43) Pub. Date: Feb. 18, 2010

(54) INTERVERTEBRAL PROSTHETIC DEVICE FOR SPINAL STABILIZATION AND METHOD OF MANUFACTURING SAME

(75) Inventor: Kent M. Anderson, Sunnyvale, CA (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/581,492

(22) Filed: Oct. 19, 2009

Related U.S. Application Data
(62) Division of application No. 11/095,214, filed on Mar. 31, 2005.

(51) Int. Cl. A61B 17/70

(52) U.S. Cl. 606/249

(57) ABSTRACT

An interspinous prosthetic device for insertion between adjacent spinous processes includes an inner/core member having upper and lower surfaces and a resiliently compressible cushion extending around at least the upper and lower surfaces of the inner member. The cushion includes at least one outwardly facing concave surface for engaging one of the spinous processes. The inner member is relatively stiff compared to the cushion.

This application is a divisional of application Ser. No. 11/095,214, filed Mar. 31, 2005, the disclosure of which is incorporated herein by reference.

### BACKGROUND

The present invention relates to an intervertebral prosthetic device for stabilizing the human spine, and a method of manufacturing same.

Spinal discs that extend between adjacent vertebrae in vertebral columns of the human body provide critical support between the adjacent vertebrae. These discs can rupture, degenerate, and/or protrude by injury, degradation, disease, or the like to such a degree that the intervertebral space between adjacent vertebrae collapses as the disc loses at least a part of its support function, which can cause impingement of the nerve roots and severe pain.

In these cases, intervertebral prosthetic devices have been designed that can be implanted between the adjacent vertebrae, both anterior and posterior of the column to prevent the collapse of the intervertebral space between the adjacent vertebrae and thus stabilize the spine.

However, many of these devices are less than optimum from a wear and strength standpoint. Also, since they are relatively stiff, they cannot flex to better accommodate the vertebrae, and do not provide a sufficient amount of shock absorption.

### SUMMARY

The intervertebral prosthetic device according to some embodiments of the invention overcomes the above deficiencies by providing increased wear, strength, and shock absorption, as well as a good fit with the anatomy.

Various embodiments of the invention may possess one or more of the above features and advantages, or provide one or more solutions to the above problems existing in the prior art.

### BRIEF DESCRIPTION OF THE DRAWINGS

FIG. 1 is a side (lateral) elevational view of an adult human vertebral column.

FIG. 2 is a posterior elevational view of the column of FIG. 1.

FIG. 3 is an enlarged, front elevational view of one of the vertebrae of the column of FIGS. 1 and 2.

FIG. 4 is an isometric view of a portion of the column of FIGS. 1 and 2, including the lower three vertebrae of the column, and depicting an intervertebral prosthetic device according to an embodiment of the invention implanted between two adjacent vertebrae.

FIG. 5 is an enlarged isometric view of the prosthetic device of FIG. 4.

FIG. 6A is a view similar to that of FIG. 5, but depicting an alternate embodiment of the prosthetic device of FIG. 5.

FIG. 6B is an enlarged isometric view depicting an element of the device of FIG. 6A.

FIG. 7 is a view similar to that of FIG. 5, but depicting an alternate embodiment of the prosthetic device of FIG. 5.

### DETAILED DESCRIPTION

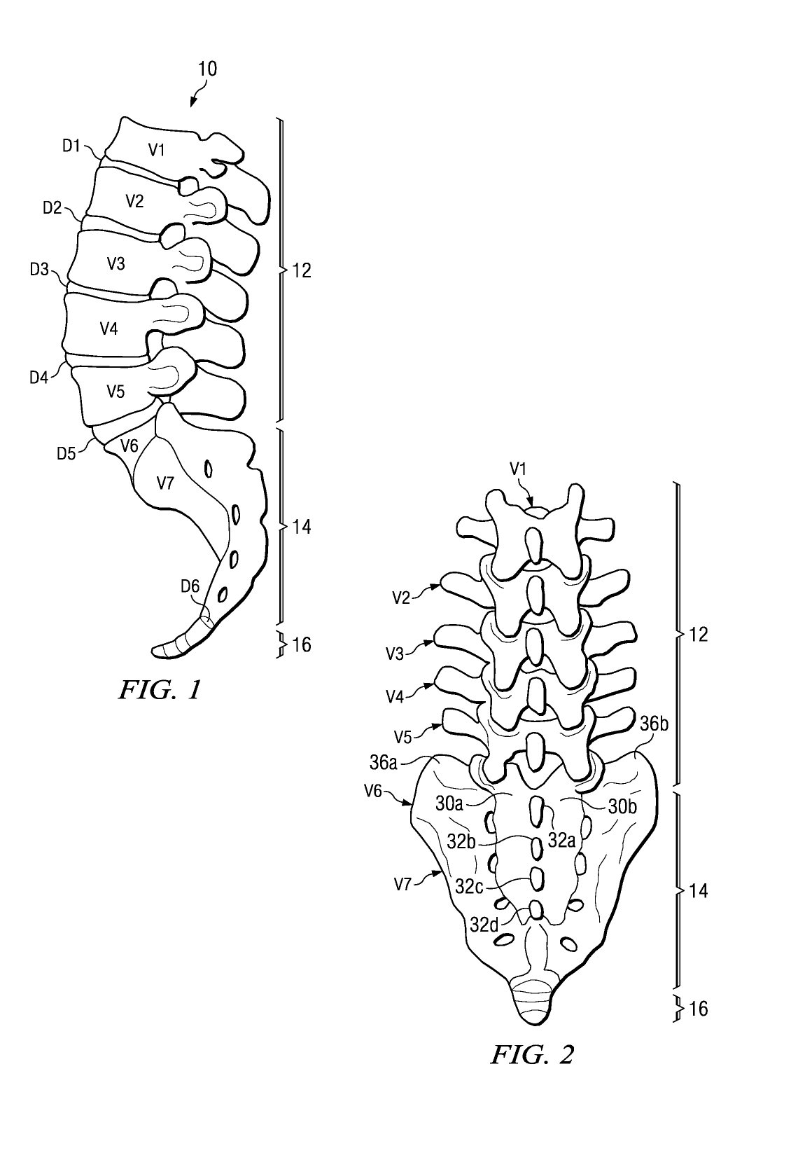

With reference to FIGS. 1 and 2, the reference numeral 10 refers, in general to a human vertebral column 10. The lower portion of the vertebral column 10 is shown and includes the lumbar region 12, the sacrum 14, and the coccyx 16. The flexible, soft portion of the vertebral column 10, which includes the thoracic region and the cervical region, is not shown.

The lumbar region 12 of the vertebral column 10 includes five vertebrae V1, V2, V3, V4 and V5 separated by intervertebral discs D1, D2, D3, and D4, with the disc D1 extending between the vertebrae V1 and V2, the disc D2 extending between the vertebrae V2 and V3, the disc D3 extending between the vertebrae V3 and V4, and the disc D4 extending between the vertebrae V4 and V5.

The sacrum 14 includes five fused vertebrae, one of which is a superior vertebra V6 separated from the vertebra V5 by a disc D5. The other four fused vertebrae of the sacrum 14 are referred to collectively as V7. A disc D6 separates the sacrum 14 from the coccyx 16 which includes four fused vertebrae (not referenced).

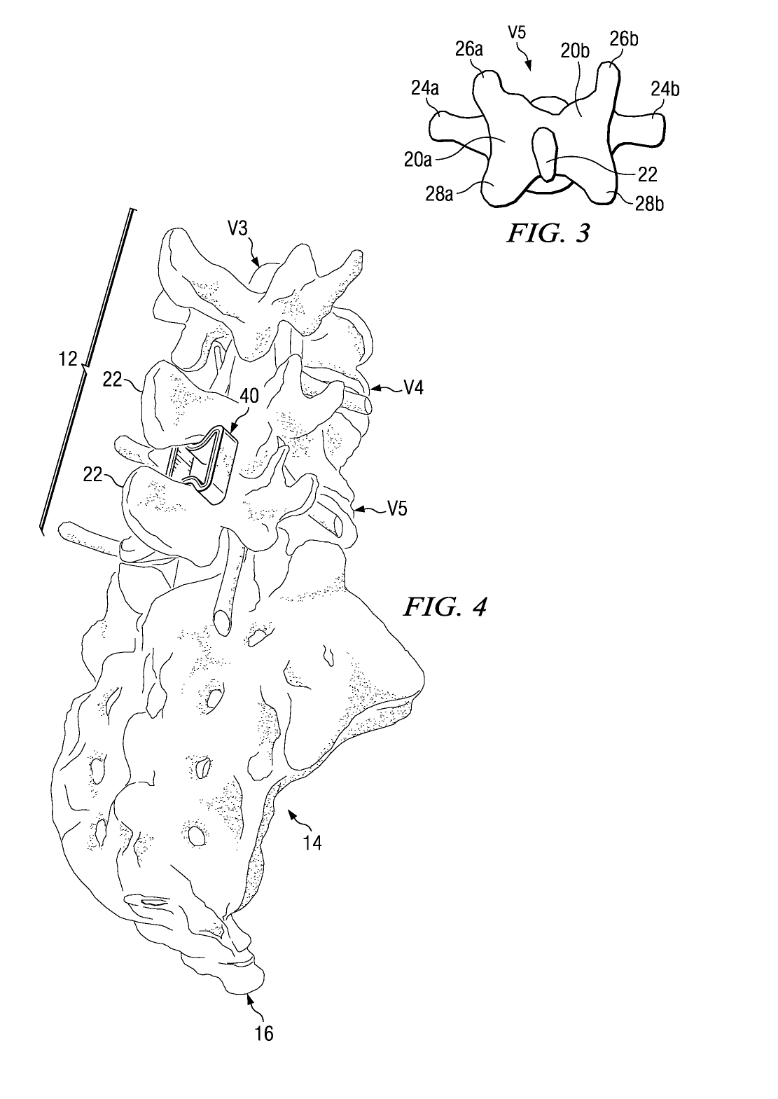

With reference to FIG. 3, the vertebra V5 includes two laminae 20a and 20b extending to either side (as viewed in FIG. 2) of a spinous process 22 that extends posteriorly from the juncture of the two laminae. Two transverse processes 24a and 24b extend laterally from the laminae 20a and 20b, respectively. Two articular processes 26a and 26b extend superiorly from the laminae 20a and 20b respectively, and two articular processes 28a and 28b extend inferiorly from the laminae 20a and 20b, respectively. The inferior articular processes 28a and 28b rest in the superior articular process of the vertebra V6 to form a facet joint. Since the other vertebrae V1-V4 are similar to the vertebra V5 they will not be described in detail.

Referring again to FIG. 2, the vertebra V6 of the sacrum 14 includes two laminae 30a and 30b extending to either side (as viewed in FIG. 2) of a median sacral crest, or spinous process, 32a that extends posteriorly from the juncture of the two laminae. The vertebra V6 also includes a pair of sacral wings 36a and 36b that extend laterally from the laminae 30a and 30b, respectively. Four additional axially-spaced sacral crests, or spinous processes, 32b-32d, are associated with the fused vertebrae V7 of the sacrum 14 and extend inferiorly from the spinous process 32a.

Referring to FIG. 4, it will be assumed that, for one or more of the reasons set forth above, the vertebrae V4 and V5 are not being adequately supported by the disc D4 and that it is therefore necessary to provide supplemental support and stabilization of these vertebrae. To this end, an intervertebral disc prosthetic device 40 according to an embodiment of the invention is implanted between the spinous processes 22 of the vertebrae V4 and V5.

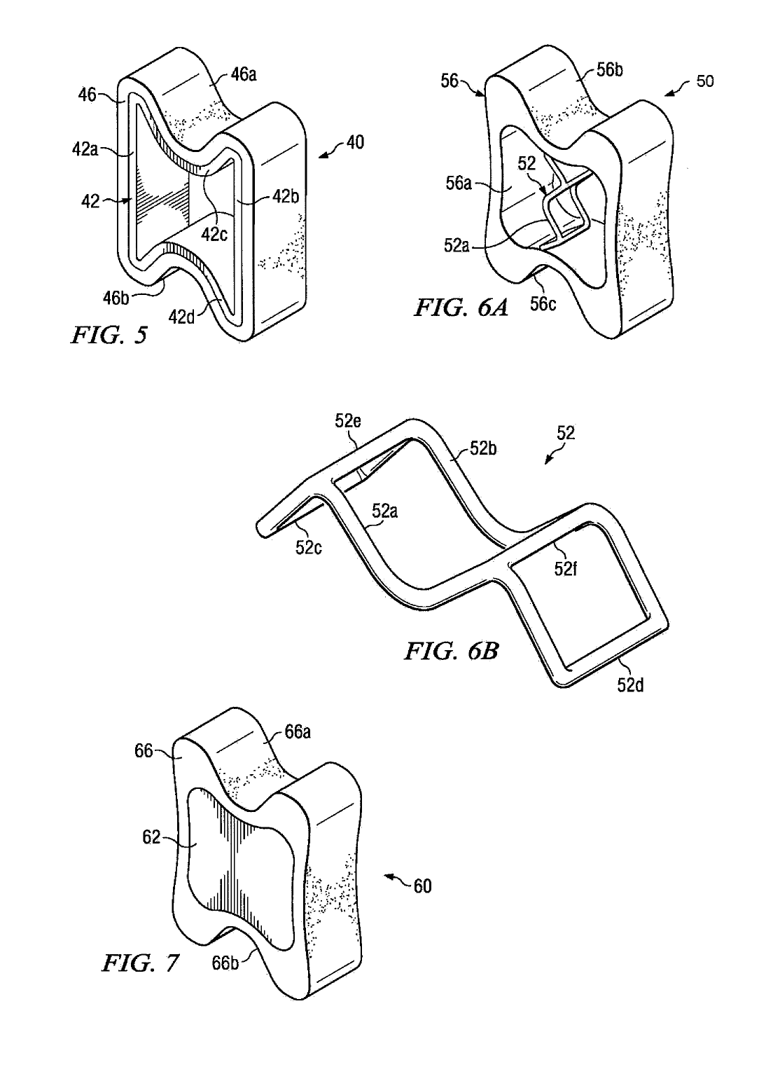

The device 40 is shown in detail in FIG. 5 and includes an inner, hollow, endoskeleton, or frame, 42 of a relatively stiff and hard material, such as metal. The frame 42 is formed by two spaced, parallel members 42a and 42b, and two additional spaced, parallel, members 42c and 42d, extending perpendicularly to the members 42a and 42b to form a closed polygon. Each member 42c and 42d is slightly curved inwardly towards the center of the frame 42, and are either formed integrally with the members 42a and 42b or are connected to the latter members in any conventional manner.

An outer layer 46 of a relatively flexible and soft material, such as silicone, is disposed around the outer surfaces of the frame 42. The layer 46 has a substantially constant thickness so that the portions 46a and 46b of the layer 46 that extend over the curved members 42c and 42d, respectively, are also curved. The layer 46 can be molded in place around the frame, and since this molding technique is conventional, it will not be described in detail.

When the device 40 is implanted between the spinous processes 22 of the vertebrae V4 and V5 as shown in FIG. 4, the processes extend in the corresponding curved portions 46a and 46b of the device. The relative stiff frame 42 adds compressive strength and durability to the device 40, while the dimensions and shape of the members 42a-42d making up the frame 42 impart a resilience, or spring-like quality, to the frame thus providing excellent shock absorption. The relatively flexible and soft layer 46 readily conforms to the processes 22 and provides additional shock absorption.

A prosthetic device according to an alternate embodiment is shown, in general, by the reference numeral 50 in FIG. 6A. The device 50 includes an inner, hollow, endoskeleton, or frame, 52 of a relatively stiff and hard material, such as metal, disposed within an over mold 56 of a relatively flexible and soft material, such as silicone.

The frame 52 is better shown in FIG. 6B and is formed by two parallel members 52a and 52b each of which is bent, or curved, in three places to form a substantially M-shaped cross section. Two spaced, parallel, cross-bars 52c and 52d extend perpendicular to, and between, the corresponding ends of the members 52a and 52b, and two spaced, parallel, cross-bars 52e and 52f also extend perpendicular to, and between, corresponding curved portions of the latter members. The cross-bars 52c, 52d, 52e, and 52f are either formed integrally with the members 52a and 52b, or are connected thereto in any conventional manner.

The over mold 56 is formed separately from the frame 52 and has an internal space 56a that receives the frame 52. The upper and lower portions 56b and 56c of the over mold 56 are curved and engage the corresponding spinous processes 22 (FIG. 4) of the vertebrae V4 and V5 when the device 60 is inserted between the processes.

When the device 50 is implanted between the spinous processes 22 (FIG. 4) of the vertebrae V4 and V5, the processes extend in the curved portions 56b and 56c. Also, the relatively stiff frame 52 adds compressive strength and durability to the device 50, while providing excellent shock absorption. The relatively flexible and soft over mold 56 readily conforms to the processes 22 and also provides additional shock absorption.

A prosthetic device according to another alternate embodiment is shown, in general, by the reference numeral 60 in FIG. 7. The device 60 includes an inner core 62 having a generally rectangular cross-section and formed of a relatively stiff material, such as hard rubber or plastic.

An outer layer 66 of a relatively flexible and soft material, such as silicone, is molded around the core 62 in a conventional manner. The upper and lower portions 66a and 66b of the layer 66 are curved and engage the corresponding processes 22 (FIG. 4) of the vertebrae V4 and V5 when the device 60 is inserted between the processes.

The device 60 could be fabricated by a two-part molding process in which the inner core 62 is initially molded of a relatively stiff, hard rubber or hard plastic. The outer layer 66, of the relatively flexible and soft material, would then be molded over the core 62.

When the device 60 is implanted between the processes 22 of die vertebrae V4 and V5 in the manner described above, the relatively stiff and hard material of the core 62 provides compressive strength and durability, while the flexible and soft layer 66 readily conforms to the processes 22 and also provides additional shock absorption.

### Variations

It is understood that variations may be made in the foregoing without departing from the invention and examples of some variations are as follows:

A core, similar to the core 62 of the embodiment of FIG. 7, but of a different shape, can be provided in the frames 42 and/or 52.

The devices of the above embodiments can be implanted between body portions other than processes of vertebrae.

The devices of the above embodiments can be inserted between two vertebrae following a corpectomy in which at least one vertebra has been removed.

The frames 42 and 52, and the core 62, may vary in shape, size, composition, and physical properties.

The outer layers 46, 56, and 66 can be formed by any suitable flexible and soft material other than silicone, can take shapes that are different than those described above, and can be secured over the frames 42 and 52, and the core 62 other than by molding.

Any spatial references made above, such as “under”, “over”, “between”, “upper”, “lower”, “top”, “bottom”, etc. are for the purpose of illustration only and do not limit the specific orientation or location of the structure described above.

The preceding specific embodiments are illustrative of the practice of the invention. It is to be understood, therefore, that other expedients known to those skilled in the art or disclosed herein, may be employed without departing from the invention or the scope of the appended claims, as detailed above. In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw are equivalent structures.

1. An interspinous prosthetic device for insertion between adjacent spinous processes, the device comprising:
an inner member having upper and lower surfaces;
a resiliently compressible cushion extending around at least the upper and lower surfaces of the inner member;
wherein said cushion includes at least one outwardly facing concave surface for engaging one of the spinous processes;
wherein the inner member is relatively stiff compared to the cushion.

2. The device of claim 1 wherein the cushion has two outwardly facing concave surfaces that face in generally opposite directions for engaging the two spinous processes respectively.

3. The device of claim 1 wherein the inner member includes first and second lateral sides extending generally perpendicular to the upper and lower surfaces and wherein the cushion is molded over the upper and lower surfaces and around first and second lateral sides of the inner member.

4. The device of claim 1 wherein the inner member comprises hard rubber or plastic.

5. The device of claim 1 wherein the cushion comprises silicone.

6. The device of claim 1 wherein the inner member includes a convex surface corresponding to the at least one outwardly facing concave surface; wherein the convex surface is in intimate contact with the cushion.

7. The device of claim 1 wherein the inner member is a solid core such that when the device is disposed between the spinous processes, a center of the core lies in a sagittal plane defined by the spinous processes.
